(19) United States
(12) Patent Application Publication
(10) Pub. No.: US 2026/0187801 A1
(43) Pub. Date: Jul. 2, 2026

(54) MEDICAL IMAGE PROCESSING DEVICE, OCT DEVICE, AND STORAGE MEDIUM STORING MEDICAL IMAGE PROCESSING PROGRAM

(71) Applicant: NIDEK CO.,LTD., Gamagori-shi (JP)

(72) Inventors: Ryosuke SHIBA, Gamagori-shi (JP); Tetsuya KANO, Gamagori-shi (JP)

(21) Appl. No.: 19/004,440

(22) Filed: Dec. 29, 2024

(51) Int. Cl. G06T 7/00; A61B 5/00; G06T 7/246; G06T 7/73; G06T 11/00

(52) U.S. Cl. CPC G06T 7/0016; A61B 5/0066; G06T 7/248; G06T 7/74; G06T 12/30; G06T 2200/24; G06T 2207/10101; G06T 2207/30041; G06T 2211/456

(57) ABSTRACT

A medical image processing device includes a control unit that executes: an image data acquisition step of acquiring first image data and second image data that were captured on a living tissue of a same subject at different times; a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and a correlation output step of processing and outputting the plurality of correlations for the positions in the X-Y directions.

### CROSS REFERENCE TO RELATED APPLICATION

This application is based on Japanese Patent Application No. 2023-059148 filed on Mar. 31, 2023 and Japanese Patent Application No. 2023-059149 filed on Mar. 31, 2023. The entire disclosure of the above applications is incorporated herein by reference.

### TECHNICAL FIELD

The present disclosure relates to a medical image processing device and an OCT device that are configured to process image data of a three-dimensional image of a living tissue, and a storage medium storing a medical image processing program executed in the medical image processing device.

### BACKGROUND

A three-dimensional images of living tissues are useful for assisting healthcare professionals in the treatment of patients. In recent years, a technique has been proposed for a user such as a medical professional to recognize the difference between a plurality of three-dimensional images (for example, changes in the living tissue over time appearing in the plurality of three-dimensional images) by processing a plurality of three-dimensional images on the same living tissue. For example, a device performs a segmentation process on a three-dimensional image of a fundus, which is an example of a living tissue, to detect a layer appearing in the three-dimensional image, and generates an analysis map showing a two-dimensional distribution of the thickness of a specific layer. In addition, a conventional device is configured to capture the same position of the same subject at different times and generate an analysis map for each of the captured plurality of three-dimensional images. The generated analysis map may be used for a user to recognize changes in the tissue over time.

### SUMMARY

However, when using segmentation processing on a three-dimensional image, it was difficult for a user to accurately recognize the difference between a plurality of three-dimensional images if the accuracy of the segmentation process on at least one of the three-dimensional images was low. In addition, it was difficult to reflect, in the results of the segmentation treatment, changes over time that have a small effect on the layer (for example, changes in vitiligo appearing in a tissue over time). The inventors of the present disclosure have newly discovered a technique different from the segmentation process in order to allow users to appropriately recognize the difference between a plurality of three-dimensional images.

One exemplary objective of the present disclosure is to provide a medical image processing device, an OCT device, and a storage medium storing a medical image processing program that are capable of allowing users to appropriately recognize the difference between a plurality of three-dimensional images.

In a first aspect of the present disclosure, a medical image processing device processes image data of a living tissue. The image data is image data of a three-dimensional image formed by arranging, in X-Y directions which are two-dimensional directions intersecting a Z direction. A plurality of Z-direction images each extends in the Z direction which is a depth direction of the living tissue. The medical image processing device includes: a control unit that is configured to execute: an image data acquisition step of acquiring first image data and second image data that were captured on a living tissue of a same subject at different times, at least some of imaging positions for the first and second image data overlapping with each other; a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and a correlation output step of processing and outputting the plurality of correlations for the positions in the X-Y directions.

In a second aspect of the present disclosure, an OCT device captures image data of a living tissue by receiving interference light between reference light and reflected light of measurement light emitted onto the living tissue. The image data is image data of a three-dimensional image formed by arranging, in X-Y directions which are two-dimensional directions intersecting a Z direction. A plurality of Z-direction images each extends in the Z direction which is a depth direction of the living tissue. The OCT device includes: a control unit that is configured to execute: an image data acquisition step of acquiring first image data and second image data that were captured on a living tissue of a same subject at different times, at least some of imaging positions for the first and second image data overlapping with each other; a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and a correlation output step of processing and outputting the plurality of correlations for the positions in the X-Y directions.

In a third aspect of the present disclosure, a non-transitory, computer readable, storage medium stores a medical image processing program for a medical image processing device that processes image data of a living tissue. The image data is image data of a three-dimensional image formed by arranging, in X-Y directions which are two-dimensional directions intersecting a Z direction. A plurality of Z-direction images each extends in the Z direction which is a depth direction of the living tissue. The program, when executed by a control unit of the medical image processing device, causes the control unit to execute: an image data acquisition step of acquiring first image data and second image data that were captured on a living tissue of a same subject at different times, at least some of imaging positions for the first and second image data overlapping with each other; a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and a correlation output step of processing and outputting the plurality of correlations for the positions in the X-Y directions.

According to the medical image processing device, the OCT device, and the storage medium storing the medical image processing program in the present disclosure, the difference between the plurality of three-dimensional images can be appropriately recognized by users.

### BRIEF DESCRIPTION OF DRAWINGS

FIG. 1 is a block diagram showing a schematic configuration of a medical image processing system 100.

FIG. 2 is an explanatory diagram for explaining a method implemented by an imaging device 1 in the present embodiment for capturing a three-dimensional image of a living tissue 50.

FIG. 3 is an explanatory diagram for explaining a state in which a three-dimensional image is being formed by a plurality of two-dimensional images 61.

FIG. 4 is a diagram illustrating one example of the three-dimensional image 60.

FIG. 5 is a view illustrating one example of a plurality of Z-direction images 62 included in the two-dimensional image 61.

FIG. 6 is a diagram illustrating one example of medical image processing executed by the medical image processing device 40.

FIG. 7 is a diagram illustrating one example of a first front image 65A generated from a three-dimensional first image 60A and a second front image 65B generated from a three-dimensional second image 60B.

FIG. 8 is an explanatory diagram for explaining a method for acquiring correlation and displacement between Z-direction images 62A and 62B at the same position in the X-Y directions.

FIG. 9 is a diagram showing one example of a similarity map 70.

FIG. 10 is a flowchart of a two-dimensional image display process executed during a medical image process.

FIG. 11 is a diagram illustrating one example of a state in which an extraction position 75 of the two-dimensional image is set on the similarity map 70.

FIG. 12 is a diagram illustrating one example of a first two-dimensional image 61A, a second two-dimensional image 61B, and a composite two-dimensional image 69.

### DESCRIPTION OF EMBODIMENTS

### Overview

A first aspect of the medical image processing device exemplified in the present disclosure processes image data of a living tissue. The image data is image data of a three-dimensional image that is formed by arranging, in a X-Y directions intersecting a Z direction, a plurality of Z-direction images each extending in the Z direction which is a depth direction of the living tissue. A control unit of the medical image processing device executes an image data acquisition step, a correlation acquisition step, and a correlation output step. At the image data acquisition step, the control unit acquires first image data and second image data that are captured at different times for a living tissue of the same subject. In the first and second image data, at least some of the imaging positions overlap with each other. At the correlation acquisition step, the control unit is configured to acquire, for positions in the X-Y directions, a plurality of correlations (may be referred to as “similarity”) between the plurality of Z-directions images in the first image data and the plurality of Z-direction images in the second image data, each of the correlations being acquired for a pair of one of the plurality of Z-direction images of the first image data and a corresponding one of the plurality of Z-direction images of the second image data both of which has a same imaging position. At the correlation output step, the control unit is configured to process and output the plurality of correlations for the positions in the X-Y directions.

According to the first aspect in the present disclosure, each of the correlations, for the positions in the X-Y directions (i.e., on the X-Y plane), between the two Z-direction images (that is, the Z-direction image in the first image data and the Z-direction image in the second image data) both of which has the same imaging position in the X-Y directions is acquired. Among the positions in the X-Y directions, at a position where there is a small difference between the first image data and the second image data (for example, a position where the change in the living tissue is small, etc.), the correlation between the two Z-direction images is high. On the contrary, among the positions in the X-Y directions, at a position where there is a large difference between the first image data and the second image data (for example, a position where the change in the living tissue is large, etc.), the correlation between the two Z-direction images is low. Therefore, a user can appropriately recognize the difference between the first image data and the second image data by checking the correlation information acquired for each of the positions in the X-Y directions. For example, by checking the correlation information acquired for each of the positions in the X-Y directions, the user can appropriately recognize the change in the living tissue over time between the time of acquiring the first image data and the time of acquiring the second image data.

In the present disclosure, image data of a three-dimensional image captured by an OCT device is processed. The OCT device can capture a three-dimensional image of a living tissue (a three-dimensional tomographic image) using the principle of optical coherence tomography. As an example, the OCT device in the present disclosure captures a three-dimensional image by performing scanning using a spot of light (measurement light) in a two-dimensional direction. However, an irradiation optical system of the OCT device may simultaneously emit measurement light onto a two-dimensional region in the living tissue of the subject. In this case, the photodetector may be a two-dimensional photodetector that detects an interference signal in the two-dimensional region in the tissue. That is, the OCT device may capture a three-dimensional image according to the principle of so-called full-field OCT (FF-OCT). Further, the OCT device may simultaneously emit the measurement light on an irradiation line extending in a one-dimensional direction in the tissue, while scanning by moving the measurement light in a direction intersecting the irradiation line. In this case, the photodetector may be a one-dimensional photodetector (for example, a line sensor) or a two-dimensional photodetector. That is, the OCT device may capture a three-dimensional image according to the principle of so-called line-field OCT (LF-OCT).

However, the techniques exemplified in the present disclosure can also be applied to processing data of images captured by an imaging device other than an OCT device. For example, the techniques exemplified in the present disclosure may be used to process image data captured by an MRI (magnetic resonance imaging) device, a CT (computed tomography) device, or the like.

In the present disclosure, a one-dimensional image extending in the Z direction along the optical axis of the light for the OCT image (a so-called A-scan image) is processed as a Z-direction image. However, the Z-direction image is not necessarily limited to the A-scan image. For example, even if the three-dimensional image is not the OCT image, a plurality of pixel sequences arranged in the Z direction may be processed as the Z-direction image. Further, a set of a plurality of pixel sequences extending in the Z direction may be processed as the Z-direction image. That is, in each Z-direction image, a plurality of pixels may be included in the X-Y directions as well.

A method for acquiring a correlation between two Z-direction images (that is, a Z-direction image in the first image data and a Z-direction image in the second image data) in which the imaging positions in the X-Y directions are the same may be appropriately selected. For example, the control unit may acquire a correlation by searching for a value of the highest correlation between the two Z-direction images while changing the relative positions in the Z direction of the two Z-direction images having the same imaging position. This method is known as template matching. In this case, the highest value of the correlation between the two Z-direction images is detected as well as the displacement in the Z direction between the two Z-direction images. Further, as a correlation acquisition method, at least one of normalized cross-correlation, correlation coefficient, phase correlation, or the like may be used. SSD (sum of squared errors), SAC (sum of absolute errors), or the like may be acquired as a correlation. In this case, the smaller the value, the higher the similarity between the two Z-direction images.

At the correlation output step, the control unit may execute a generation process to generate a similarity map that shows a two-dimensional distribution in the X-Y directions of the strength of the correlations acquired for the positions in the X-Y directions. Then, the control unit may output the generated similarity map.

In this case, by checking the output similarity map, a user can intuitively recognize the two-dimensional distribution in the X-Y directions of the strength of the correlations (that is, the degree of the similarity) between the first image data and the second image data. For example, by checking the output similarity map, a user can appropriately recognize the positions in the X-Y directions at which the living tissue is likely to have a large change over time.

The control unit may further execute an extraction position reception step and an extraction display step. At the extraction position reception step, the control unit accepts an instruction for specifying the extraction position of the two-dimensional image from a user on the similarity map displayed on the display. At the extraction display step, when an instruction for specifying the extraction position is received at the extraction position reception step, the control unit extracts a two-dimensional image that passes through the specified extraction position and spreads in the Z direction from at least one of the first and second image data and displayed the extracted image on the display.

In this case, the user can have a two-dimensional image at an appropriate position displayed after recognizing the two-dimensional distribution of the strength of the correlations between the first image data and the second image data shown by the similarity map. For example, at the positions where the correlation indicated by the similarity map is low, there is a high possibility that the greater change in the living tissue over time occurs at the position between the first image data and the second image data. Therefore, by specifying the extraction position at a position where the correlation is low in the similarity map, the user can appropriately recognize, based on the two-dimensional image, the change in the living tissue over time. Furthermore, the user can recognize the position at which the two-dimensional image is extracted on the similarity map. Therefore, the efficiency of medical treatment can be improved.

Note that a specific method for receiving an instruction to specify the extraction position from a user can be appropriately selected. For example, the instruction for specifying the extraction position may be accepted by allowing the user to specify a linear-shaped extraction position on the two-dimensional similarity map. In this case, a user may specify a linear-shaped extraction position (in other words, a linear-shaped area) by operating an operation unit (for example, at least one of a mouse, a touch panel, etc.). The shape of the line indicating the extraction position may be various shapes such as a straight line, a curved shape, and an annular shape. Needless to say, a plurality of extraction positions may be specified. Further, a user may specify a linear-shaped extraction position in an arbitrary shape on the similarity map by operating at least one of a mouse and a touch panel (for example, dragging a mouse).

At the extraction display step, the control unit may extract two two-dimensional images each of which passes through the extraction position specified at the extraction position reception step and spreads in the Z direction from the first and second image data and display the extracted images on the display.

In this case, a user can easily compare the two-dimensional image in the first image data and the two-dimensional image in the second image data at the same extraction position specified based on the strength of the correlations between the first image data and the second image data. For example, by specifying the extraction position at a position where the correlation is low in the similarity map and comparing the displayed two two-dimensional images, the user can appropriately recognize the change in the living tissue over time.

Note that the method for displaying the two two-dimensional images extracted at the same position of the first image data and the second image data can be appropriately selected. For example, the control unit may display the two extracted two-dimensional images side by side on the display unit, or may alternately display the two two-dimensional images on the display unit by switching. Further, the control unit may combine and display the two extracted two-dimensional images by a method described later.

The control unit may further execute an extraction position automatic setting step and an extraction display step. At the extraction position automatic setting step, the control unit automatically sets the extraction position of the two-dimensional image to a position that includes a point having the lowest correlation on the similarity map. At the extraction display step, the control unit may extract a two-dimensional image that passes through the extraction position automatically set at the extraction position automatic setting step and spreads in the Z direction from at least one of the first and second image data and display the extracted image on the display.

In this case, a user can easily check the two-dimensional image at the position where the correlation between the first image data and the second image data shown by the similarity map is lowest. For example, at the position where the correlation indicated by the similarity map is lowest, there is a high possibility that the greater change in the living tissue over time occurred between the first image data and the second image data. Therefore, the efficiency of follow-up observation by a user can be improved.

As described above, the extraction position may be a linear shape. The shape of the line indicating the extraction position may be various shapes such as a straight line, a curved shape, and an annular shape. Multiple extraction positions may be specified

A specific method for automatically setting the extraction position for the two-dimensional image can be appropriately selected. For example, the control unit may search for a position where the cumulative value of correlation at the extraction position of a predetermined shape (for example, a line, etc.) is lowest, and automatically set the searched position as the extraction position. Further, the control unit may automatically set the extraction position at a linear position (or a linear area) having a center at the position where the correlation is lowest and extending in a predetermined direction (for example, in the X direction or the Y direction, etc.). The control unit may automatically set the extraction position to a linear position that passes through both the position with the lowest correlation and a reference position on the image (for example, macular center or papillary center, etc.).

At the extraction display step, the control unit may extract two-dimensional images each of which passes through the extraction position automatically set at the extraction position automatic setting step and spreads in the Z direction from the first and second image data and display the extracted images on the display.

In this case, a user can easily compare the two-dimensional image in the first image data and the two-dimensional image in the second image data at the same extraction position including a point with the lowest correlation between the first image data and the second image data. Therefore, the user can be appropriately assisted in medical treatment. As described above, various methods can be selected as a method for displaying the two two-dimensional images.

At the correlation output step, the control unit may execute a process of integrating a plurality of correlation values acquired for the positions in the X-Y directions, and output the calculated correlation integration value.

In this case, the user can appropriately recognize the difference between the first image data and the second image data by the correlation integration value of the correlations. For example, the user can recognize, by the integrated value of the correlations, the degree of overall change in the living tissue over time between the time at which the first image data was captured and the time at which the second image data was captured. Further, it is also possible to evaluate, by the integrated value of the correlations, the accuracy of the alignment of the first image data and the second image data when the correlation between the Z direction images is acquired. Further, if the accuracy of imaging of at least one of the first image data and the second image data is low, the integrated value of the correlations is small. Therefore, it is also possible to evaluate the accuracy of imaging the image by the integrated value of the correlations. For example, when the second image data is newly captured, if the integration value of the correlations between the first image data and the second image data is less than a threshold value, the control unit determines that the accuracy of imaging the second image data is low. Then, the control unit may output an instruction for capturing the second image data again.

Note that a method for integrating a plurality of correlation values can be selected as appropriate. For example, the control unit may integrate the values of a plurality of correlations by calculating the average value, the median, maximum, minimum, and the like of the plurality of correlations. Further, the control unit may calculate, as an integrated value, the number or percentage of correlations each having a value greater than the threshold value, or the number or percentage of correlations each having a value less than the threshold value.

The control unit may further execute a first front image generation step and a second front image generation step. At the first front image generation step, the control unit generates, based on the first image data, a two-dimensional first front image that is an image of the target subject when viewed in the Z direction. At the second front image generation step, the control unit generates, based on the second image data, a two-dimensional second front image that is an image of the target subject when viewed in the Z direction. At the correlation acquisition step, the control unit may acquire each of the correlations by identifying the pair of the Z-direction image of the first image data and the Z-direction image of the second image data in which the imaging positions of the living tissues in the X-Y directions are the same based on the first front image and the second front image.

In this case, the front images are generated from the first image data and the second image data for which the same position in the X-Y directions needs to be specified, and then the same position is specified based on the generated front images. Therefore, compared to another method (for example, a method for specifying a same position based on front images captured by a principle different from the principle of capturing the first image data and the second image data), the same position of the first image data and the second image data in the X-Y directions can be identified more accurately.

A specific method for generating a front image (the first front image and the second front image) based on image data (the first image data and the second image data) can be appropriately selected. For example, the control unit may generate the front image by adding (or may be an additive average) the pixel values of a plurality of pixels arranged in the Z direction for each of the plurality of Z-direction images in the image data. Further, the control unit may generate the front image (so-called, Enface images) based on pixel values at a specific position or range (for example, pixel values of a specific layer, pixel values of a specific boundary, among the layers and boundaries appearing in the image data, or pixel values within a specific layer/boundary range). In this case, the control unit may execute a segmentation process to detect at least any of the layers/boundaries appearing in the image data, and generate the front images based on the results of the segmentation process.

The control unit may specify the Z-direction image of the first image data and the Z-direction image of the second image data in which the imaging positions of the living tissues in the X-Y directions are the same by aligning the first front image and the second front image with each other. In this case, by aligning the first front image and the second front image, the positions of the Z direction image of the first image data and the Z direction image of the second image data are aligned with each other in all ranges in the XY direction. Thus, the same positions of the first image data and the second image data in the X-Y directions can be more easily and accurately identified. When aligning the first front image and the second front image, the control unit may change the relative position and the relative angle between the two front images while overlapping the first front image and the second front image. The control unit may align the two front images by searching for a position and angle at which the correlation between the two front images has the highest value. Further, by using a method of an image local feature (for example, SIFT: Scale-Invariant Feature Transform) or of a rotational invariant phase limited correlation, the first front image and the second front image may be aligned.

Further, the control unit may specify the same position in the X-Y directions of the first image data and the second image data without actually aligning the first front image and the second front image. For example, the control unit may calculate the displacement between the living tissue appearing in the first front image and the living tissue appearing in the second front image, and based on the calculated displacement, may specify the Z-direction image of the first image data and the second image data where the imaging positions of the living tissues in the X-Y directions are the same.

The method for specifying the same position in the X-Y directions of the first image data and the second image data may be changed. For example, when capturing the first image data, a front image having the same imaging position as the first image data may be captured by a principle different from the principle of imaging the first image data. Similarly, when capturing the second image data, a second image having the same imaging position as the second image data may be captured by a principle different from the principle of imaging the second image data. The control unit may specify the same position in the X-Y directions of the first image data and the second image data based on the front image captured by a principle different from the principle of imaging the first image data and the second image data.

The control unit may further execute a first image alignment step and a second image alignment step. At the first image alignment step, the control unit performs alignment in the Z direction between the plurality of Z-direction images in the first image data. At the second image alignment step, the control unit performs alignment in the Z direction between the plurality of Z-direction images in the second image data.

At least one of the plurality of Z-direction images 62 included in the three-dimensional image data may include a Z-direction image in which the position of the living tissue in the Z direction is deviated from the normal position due to the movement of the living tissue during imaging, vibration of the device, and the like. By aligning between the plurality of Z-direction images, various effects due to images whose positions in the Z direction are deviated from the normal position are reduced. For example, when a two-dimensional image is extracted from at least one of the first image data and the second image data, the displacement in the Z direction of each Z-direction image in the extracted two-dimensional image is suppressed. Thus, the two-dimensional image can be appropriately extracted.

At the correlation acquisition step, the control unit may acquire the correlations at the positions in the X-Y directions between the entire first image data and the entire second image data. In this case, the difference between the first image data and the second image data is appropriately recognized throughout the imaging area of the images. Therefore, the user can be appropriately assisted in medical treatment.

However, the control unit may also acquire correlations at the positions in the X-Y directions between some regions overlapped with each other among the first image data and the second image data.

The date on which the first image data was captured and the date on which the second image data was captured may be different. In this case, the changes in the living tissue between the two capturing timings of the first image data and the second image data is easily recognized. Therefore, follow-up observation on the living tissue and the like can be more appropriately performed.

The control unit may acquire a probability distribution for identifying at least one of the layers or the boundary of a fundus tissue appearing in the three-dimensional image by inputting at least one of the first image data and the second image data into a mathematical model trained by a machine learning algorithm. Then, the control unit may generate a deviation map based on the acquired probability distribution. The deviation map shows a two-dimensional distribution of the degree of deviation of the actually acquired probability distribution with respect to the probability distribution that would be acquired when the layer or boundary to be identified is accurately identified. The control unit may display the deviation map and the similarity map on the display unit at the same time or by switching.

As a premise, if there is no abnormality in the structure of the layer/boundary, it is easy to accurately identify the layer/boundary by the mathematical model, since the probability distribution for the layer/boundary identified by the mathematical model is likely to be biased. On the contrary, if there is an abnormality in the structure of the layer/boundary, the probability distribution is less likely to bias. Therefore, the degree of anomaly of the structure of the layer/boundary is likely to appear in the deviation map that shows the two-dimensional distribution of the degree of deviations between the probability distribution that would be acquired when the layer/boundary is accurately identified and the probability distribution that was actually acquired. Further, as described above, according to the similarity map, the position in the X-Y directions, which is likely to have a large change over time in the living tissue, is easily recognized. Therefore, the deviation map and the similarity map are displayed on the display unit at the same time or switched, making it easier for the user to more appropriately grasp the state of the subject's living tissue. In addition, the control unit may decide whether to simultaneously display the deviation degree map and the similarity map, whether to perform a switching display of the deviation degree map and the similarity map, or the like according to an instruction input by a user. In this case, since the map desired by a user is appropriately displayed on the display unit, the efficiency of medical treatment by the user can be appropriately improved.

The control unit may generate a thickness map indicating a two-dimensional distribution of the analysis results for the thickness of at least one of layers in the fundus tissue appearing in at least one of the first image data and the second image data. The control unit may display the thickness map and the similarity map to the display unit at the same time or by switching.

According to the thickness map, the distribution of the thickness of the layers of the living tissue is properly recognized. Further, as described above, according to the similarity map, the position in the X-Y directions, which is likely to have a large change over time in the living tissue, is easily recognized. Therefore, the thickness map and the similarity map are displayed on the display unit at the same time or switched, making it easier for the user to more appropriately recognize the state of the subject's living tissue. In addition, the control unit may decide whether to simultaneously display the thickness map and the similarity map, whether to perform a switching display of the thickness map and the similarity map, or the like according to an instruction input by a user. In this case, since the map desired by a user is appropriately displayed on the display unit, the efficiency of medical treatment by the user can be appropriately improved.

Furthermore, the control unit may display at least two of the deviation map, the thickness map, and the similarity map on the display unit simultaneously or by switching. Further, a map showing a two-dimensional distribution of information on a living tissue other than the deviation map and the thickness map generated based on at least one of the first image data and the second image data may be displayed simultaneously with the similarity map or by switching between the map and the similarity map.

A second aspect of the medical image processing device exemplified in the present disclosure processes image data of living tissues. The control unit of the medical image processing device executes a two-dimensional image acquisition step and a composite display step. At the two-dimensional image acquisition step, the control unit acquires a first two-dimensional image and a second two-dimensional image, which are images on the same type of living tissues. At the composite display step, the control unit combines the first two-dimensional image and the second two-dimensional image in a state where the two images are overlapped with each other and displays the combined image on the display unit.

According to the second aspect of the present disclosure, a first two-dimensional image and a second two-dimensional image of the same type of living tissues are superimposed and displayed on the display unit. Therefore, by confirming the two-dimensional image (hereinafter, referred to as a “composite two-dimensional image”) displayed on the display unit, a user can recognize the difference between the living tissues appearing in the first and second two-dimensional images.

At the composite display step, the control unit may combine and display the first two-dimensional image and the second two-dimensional image in different colors from each other. In this case, in one composite two-dimensional image, the subject appearing in the first two-dimensional image and the subject appearing in the second two-dimensional image are displayed in different colors. Therefore, the user can recognize, based on the composite two-dimensional image, the change in the living tissue over time between the time of capturing the first image data and the time of capturing the second image data.

At the composite display step, the control unit may set a background color, which is the color of background pixels in which the subject does not appear, to a black or white color. In the first two-dimensional image, the color of the pixel of part where the subject appears is defined as a first color. In the second two-dimensional image, the color of the pixel of part where the subject appears is defined as a second color. The control unit may set the combination of the first color and the second color to a combination that will be a color opposite to the background color (black or white) when the first and second colors are combined.

In this case, at a position where the subject of the first two-dimensional image and the subject of the second two-dimensional image overlap with each other, the subject is displayed in a color (white or black) opposite to the background color. That is, at a position where the subject of the first two-dimensional image and the subject of the second two-dimensional image overlap with each other, the subject is displayed in grayscale. Further, at a position where the subject of the first two-dimensional image and the subject of the second two-dimensional image do not overlap with each other, the subject of the first two-dimensional image is displayed in the first color, and the subject of the second two-dimensional image is displayed in the second color. As a result, at a position where the subject of the first two-dimensional image and the subject of the second two-dimensional image do not overlap with each other, the subject of each two-dimensional image is appropriately recognized with the first and second colors other than grayscale. Therefore, the user can intuitively recognize the portion where only the subject of the first two-dimensional image appears, the portion where only the subject of the second two-dimensional image appears, the part where the subject of the first two-dimensional image and the subject of the second two-dimensional image overlap with each other, and the background portion.

In other words, if the composite color of the first color and the second color does not turn to be an opposite color to the background, the first color, the second color, and the composite color are to be included in the composite two-dimensional image. Thus, the visibility of the subject is likely to decrease. For example, if the first color is red and the second color is green, yellow, which is a composite color of red and green, would be also included in the composite two-dimensional image. On the contrary, by using the composite color of the first color and the second color which is an opposite color to the background color, the position where the two subjects overlap with each other is displayed in grayscale, and the visibility of the subject can be improved.

The combination of the first and second colors can be selected as appropriate. For example, when the background color is black, the control unit may use a combined color of two of the three primary colors (Red, Green, Blue) of light that will turn to be white when all colors are combined as one of the first and second colors and may use a remaining color as the other of the first and second colors. As an example, when the background color is black, the control unit may use Magenta formed by combining Red and Blue as the first color, and Green as the second color as the remaining primary color. Further, when the background color is white, the control unit may use a combined color of two of the three primary colors (Cyan, Magenta, Yellow) of light that will turn to be black when all colors are combined as the first color and use a remaining color as the second color.

However, it is also possible to express the position where the subject of the first two-dimensional image and the subject of the second two-dimensional image overlap with each other in a color other than grayscale (that is, white or black). Even in this case, by setting the first and second colors to different colors, the user can appropriately recognize the portion where only the subject of the first two-dimensional image appears, the portion where only the subject of the second two-dimensional image appears, the part where the subject of the first two-dimensional image and the subject of the second two-dimensional image overlap with each other, and the background portion.

The control unit may execute a two-dimensional image similarity map output step. During the step, the control unit calculates a similarity between partial images where the first two-dimensional image and the second two-dimensional image are at the same position by alignment among a plurality of partial images constituting each of the first and second two-dimensional images. Then, the control unit outputs a two-dimensional image similarity map showing a two-dimensional distribution of between the calculated multiple similarities. In this case, by checking the output two-dimensional image similarity map, the user can intuitively recognize the distribution of similarity when alignment of the first two-dimensional image and the second-dimensional two-dimensional image is performed. Therefore, the user can better recognize the change in the living tissue over time between the time of capturing the first image data and the time of capturing the second image data.

Note that the partial image that serves as a unit for acquiring similarity between the first two-dimensional image and the second two-dimensional image may be pixels constituting each image. In this case, since the distribution of similarity between the two images is shown for each pixel, changes in a living tissue over time can be recognized in detail. However, the unit of the partial image may be changed. For example, similarity may be acquired using a rectangular partial image containing a plurality of pixels as a unit. Further, similarity may be acquired as a partial image of a Z-direction image which is a set of a plurality of pixels extending in the Z direction.

A specific method for generating the two-dimensional image similarity map can be appropriately selected. For example, the two-dimensional image similarity map may be generated by using a known SSIM (Structural Similarity). In this case, the SSIM is calculated in a small area including a plurality of pixels. A plurality of SSIMs are calculated while shifting the relative positions of the two sub-regions, and the average value of the calculated plurality of SSIMs is considered to be the degree of similarity. As a result, in addition to the information of a pair of partial images at the same position (for example, luminance and contrast, etc.), information on the surrounding partial images is also used, so that the difference in the structure of the image can be evaluated.

The image data may be image data of a three-dimensional image that spreads in Z direction that is the depth direction of the living tissue and the two-dimensional X-Y directions that intersects Z direction. The control unit may further execute an image data acquisition step and an extraction position setting step. At the image data acquisition step, the control unit acquires first image data and second image data that are captured at different times for a living tissue of the same subject. In the first and second image data, at least some of the imaging positions overlap with each other. At the extraction position setting step, the control unit sets an extraction position for extracting a two-dimensional image within an imaging range in the X-Y directions common to the first image data and the second image data. At the two-dimensional image acquisition step, the control unit may extract a two-dimensional image at the same extraction position in which the position in a living tissue appearing in the image is common to each other among the first image data and the second image data. At the composite display step, the control unit may combine the first two-dimensional image extracted at the extraction position of the first image data and the second two-dimensional image extracted at the extraction position of the second image data after the first and second two-dimensional images are aligned with each other. Then, the control unit may display the first and second two-dimensional images on the display unit.

In this case, two-dimensional images (that is, a first two-dimensional image and a second-dimensional image) are automatically extracted at the same position in the living tissue appearing in the image of each of the first image data and the second image data. The extracted first two-dimensional image and the extracted second two-dimensional image are combined and displayed on the display unit after the first and second images are aligned with each other. Therefore, by confirming the two-dimensional image (hereinafter, referred to as a “composite two-dimensional image”) displayed on the display unit, a user can compare living tissues appearing in the first and second two-dimensional images having the common imaging position in the living tissues. Therefore, the user can recognize, based on the composite two-dimensional image, the change in the living tissue over time between the time of capturing the first image data and the time of capturing the second image data.

In the present disclosure, image data of a three-dimensional image captured by an OCT device is processed. The OCT device can capture a three-dimensional image of a living tissue (a three-dimensional tomographic image) using the principle of optical coherence tomography. As an example, the OCT device in the present disclosure captures a three-dimensional image by performing scanning using a spot of light (measurement light) in a two-dimensional direction. However, an irradiation optical system of the OCT device may simultaneously emit measurement light onto a two-dimensional region in the living tissue of the subject In this case, the photodetector may be a two-dimensional photodetector that detects an interference signal in the two-dimensional region in the tissue. That is, the OCT device may capture a three-dimensional image according to the principle of so-called full-field OCT (FF-OCT). Further, the OCT device may simultaneously emit the measurement light on an irradiation line extending in a one-dimensional direction in the tissue, while scanning by moving the measurement light in a direction intersecting the irradiation line. In this case, the photodetector may be a one-dimensional photodetector (for example, a line sensor) or a two-dimensional photodetector. That is, the OCT device may capture a three-dimensional image according to the principle of so-called line-field OCT (LF-OCT).

However, the techniques exemplified in the present disclosure can also be applied to processing data of images captured by an imaging device other than an OCT device. For example, the techniques exemplified in the present disclosure may be used to process image data captured by an MRI (magnetic resonance imaging) device, a CT (computed tomography) device, or the like.

The method for acquiring the first two-dimensional image and the second two-dimensional image in the two-dimensional image acquisition step may be changed. The control unit may execute an image data acquisition step to acquire image data of a three-dimensional image of the living tissue that spreads in Z direction that is the depth direction of the living tissue and two-dimensional X-Y directions that intersects the Z direction. At the two-dimensional image acquisition step, the control unit may extract two-dimensional images at different extraction positions in which the positions in living tissues appearing in the images are different from each other among acquired single three-dimensional image. At the composite display step, the control unit may combine the first two-dimensional image and the second two-dimensional image in a state where the two images are overlapped with each other and may display the combined image on the display unit. In this case, the first and second two-dimensional images that are extracted at different positions in one three-dimensional image are combined and displayed. Therefore, a user can appropriately recognize, based on the composite two-dimensional image, the difference between the first two-dimensional image and the second dimensional image in which the imaging positions (the extraction positions) are different from each other in one living tissue.

A specific method for extracting a two-dimensional image at each of the two extraction positions of the image data in a single three-dimensional image can be appropriately selected. For example, the control unit may specify the position of a specific reference site (for example, macula or papillae, etc.) and set the extraction position of the two-dimensional image at two positions that are symmetrical with the specified reference site. In this case, the difference between the two-dimensional images of the two positions symmetrical with the reference site is appropriately compared by the composite two-dimensional image. Specifically, the control unit may set a macula as a reference site and set the extraction position of the two-dimensional image on two linear-shaped positions that are upper and lower parts and are symmetrical with the reference site. In diseases such as glaucoma, the symmetry of the upper and lower positions of the tissue relative to the macula is often effectively used for medical treatment. Therefore, the difference between the two-dimensional images of the upper and lower positions that are symmetrical with the macula is appropriately compared using the composite two-dimensional image. As a result, medical treatment can be more appropriately supported.

Further, the control unit may acquire a first two-dimensional image and a second dimensional image that were captured at different times at the same position of a living tissue of the same subject at the two-dimensional image acquisition step. At the composite display step, the control unit may combine the first two-dimensional image and the second two-dimensional image in a state where the two images are overlapped with each other and may display the combined image on the display unit. That is, the control unit does not perform a process of extracting a two-dimensional image at the same position from two three-dimensional images captured at different times for a living tissue of the same subject, and the first two-dimensional image and the second two-dimensional image captured at the same position at different times may be directly acquired. Even in this case, a user can recognize, based on the composite two-dimensional image, the change in the living tissue over time between the time of capturing the first two-dimensional image and the time of capturing the second two-dimensional image.

In the present disclosure, a two-dimensional tomographic image spreading in a depth direction of a tissue is used as the first two-dimensional image and the second two-dimensional image each of which is a target to be combined is mainly described. However, the first two-dimensional image and the second two-dimensional image each of which is the target to be combined may be two-dimensional front images that are captured when the tissue is viewed from a front side. In this case, the difference between the front images of the living tissue appearing in the first and second two-dimensional images is appropriately recognized using the composite two-dimensional image. The method for acquiring the first two-dimensional image and the second two-dimensional image, which are two-dimensional front images, can also be appropriately selected. For example, the two-dimensional front image may be acquired by an imaging device that captures a two-dimensional front image of a living tissue (if the living tissue is a fundus, at least one of a fundus camera and a scanning laser ophthalmoscope, etc.). Further, the control unit may generate the front images (so-called, Enface images) based on pixel values at a specific position or range (for example, pixel values of a specific layer, pixel values of a specific boundary, among the layers and boundaries appearing in the three-dimensional image, or pixel values within a specific layer/boundary range).

In the present disclosure, the first two-dimensional image and the second two-dimensional image captured for a living tissue of the same subject are combined and displayed. However, the control unit may combine and display the first two-dimensional image and the second two-dimensional image captured for the same type of living tissues of a plurality of subjects. In this case, the difference in the living tissues of the plurality of subjects is easily recognized appropriately by the combined two-dimensional image. Further, the control unit may combine and display a first two-dimensional image of the left eye and a second image of the right eye of the same subject. In this case, the first two-dimensional image and the second two-dimensional image may be combined at positions that are symmetrical to each other in the living tissues of the left eye and the right eye. In addition, when the left and right sides of a living tissue appearing in each of the first and second two-dimensional images are substantially symmetrical, the two images may be combined after the left side or the right side of one image was determined. In this case, it is easy to properly compare the living tissues of the left eye and the right eye.

The control unit may further execute a first front image generation step and a second front image generation step. At the first front image generation step, the control unit generates, based on the first image data, a two-dimensional first front image that is an image of the target subject when viewed in the Z direction. At the second front image generation step, the control unit generates, based on the second image data, a two-dimensional second front image that is an image of the target subject when viewed in the Z direction. At the two-dimensional image acquisition step, the same extraction position in which the position in a living tissue is common to each of the first image data and the second image data may be specified based on the first front image and the second front image.

In this case, the front images are generated from the first image data and the second image data from which the same extraction position in the X-Y directions needs to be specified, and then the extraction position of the two-dimensional image is specified based on the generated front images. Therefore, as compared to another method (for example, a method for specifying an extraction position based on front images captured by a principle different from the principle of capturing the first image data and the second image data), the extraction position for the two-dimensional image of the first image data and the second image data in the X-Y directions can be identified more accurately.

A specific method for generating a front image (the first front image and the second front image) based on image data (the first image data and the second image data) can be appropriately selected. For example, the control unit may generate the front image by adding (or may be an additive average) the pixel values of a plurality of pixels arranged in the Z direction for each of the plurality of Z-direction images in the image data. Further, the control unit may generate the front image (so-called, Enface images) based on pixel values at a specific position or range (for example, pixel values of a specific layer, pixel values of a specific boundary, among the layers and boundaries appearing in the image data, or pixel values within a specific layer/boundary range). In this case, the control unit may execute a segmentation process to detect at least any of the layers/boundaries appearing in the image data, and generate the front images based on the results of the segmentation process.

The control unit may specify the same extraction position in the first image data and the second image data in the X-Y directions by aligning the first front image and the second front image with each other. In this case, by aligning the first front image and the second front image, the positions of the first image data and the second image data on the living tissue are aligned with each other in all ranges in the XY direction. Thus, the extraction positions of the first image data and the second image data can be more easily and accurately identified. When aligning the first front image and the second front image, the control unit may change the relative position and the relative angle between the two front images while overlapping the first front image and the second front image. The control unit may align the two front images by searching for a position and angle at which the correlation between the two front images has the highest value.

Further, the control unit may specify the same extraction position in the first image data and the second image data without actually aligning the first front image and the second front image. For example, the control unit may calculate the displacement between the living tissue captured in the first front image and the living tissue captured in the second front image, and specify the extraction position based on the calculated displacement.

The method for specifying the extraction position of the two-dimensional image of the first image data and the second image data can be changed. For example, when capturing the first image data, a front image having the same imaging position as the first image data may be captured by a principle different from the principle of imaging the first image data. Similarly, when capturing the second image data, a second image having the same imaging position as the second image data may be captured by a principle different from the principle of imaging the second image data. The control unit may specify the extraction position of each of the two-dimensional images of the first image data and the second image data based on the front image captured by a principle different from the principle of imaging the first image data and the second image data.

At the composite display step, the control unit combines the first two-dimensional image and the second two-dimensional image and displays them on the display unit while the images appearing in the first two-dimensional image and the second two-dimensional image are aligned with each other in the Z direction. At least one of the plurality of Z-direction images (images extending in the Z direction) included in the two-dimensional image may include a Z-direction image in which the position of the living tissue in the Z direction is deviated from the normal position due to the movement of the living tissue during imaging, vibration of the device, and the like. Even if two two-dimensional images are combined in a state where the Z-direction images whose positions are offset from each other in the Z direction are included in the two-dimensional images, it would be difficult for a user to recognize the difference in living tissues using the composite two-dimensional image. On the contrary, since the composite two-dimensional image is displayed in a state where the images are aligned with each other in the Z direction, the subjects of the first two-dimensional image and the second two-dimensional image can be more appropriately compared using the composite two-dimensional image.

The control unit may extract the first two-dimensional image and the second two-dimensional image after aligning in the Z direction in advance between the first image data and the second image data, each of which is a three-dimensional image. In this case, regardless of the extraction position, the relative positions of the extracted first two-dimensional image and the second two-dimensional image in the Z direction are appropriately fixed.

A method for aligning in the Z direction between the first image data and the second image data can also be appropriately selected. For example, the control unit may search for a value of the highest correlation between the two Z-direction images while changing the relative positions in the Z direction of the two Z-direction images (that is, the Z-direction image in the first image data and the Z-direction image in the second image data) having the same imaging position in the X-Y directions. The control unit may align the two Z-direction images based on the displacement in the Z direction when the searched correlation has the highest value. Here, before performing alignment in the Z direction between the first image data and the second image data, the control unit may perform, in advance, alignment in the Z direction between the plurality of Z-direction images included in the first image data and alignment in the Z direction between the plurality of Z-direction images included in the second image data. In this case, even if there is a portion where the position in the Z direction deviates from the normal position due to the movement of the living tissue during imaging, it is easy to perform alignment in the Z direction with higher accuracy.

However, the control unit may also perform alignment in the Z direction between the first two-dimensional image and the second two-dimensional image after acquiring (for example, extracting) the first two-dimensional image and the second two-dimensional image.

The first and second dimensional images may be images taken on the living tissue of the same subject. The date on which the first two-dimensional image was taken and the date on which the second two-dimensional image was taken may be different. In this case, the change in the living tissue between the two capturing timings of the first two-dimensional image and the second two-dimensional image is easily recognized. Therefore, follow-up observation on the living tissue and the like can be more appropriately performed.

### Embodiment

Hereinafter, one of exemplary embodiments according to the present disclosure will be described. In the present embodiment, three-dimensional image data of a fundus tissue of a subject eye E captured by an OCT device is processed. However, the images processed by the technique of the present disclosure may be images of a tissue other than the fundus tissue. For example, the processing target image may be an image of a living tissue other than the fundus of the subject eye E (for example, the anterior segment of the eye), or an image of a living tissue other than the subject eye E (for example, skin, digestive organs, or brain, etc.). Further, as described above, an imaging device for capturing images to be processed is not necessarily limited to the OCT device.

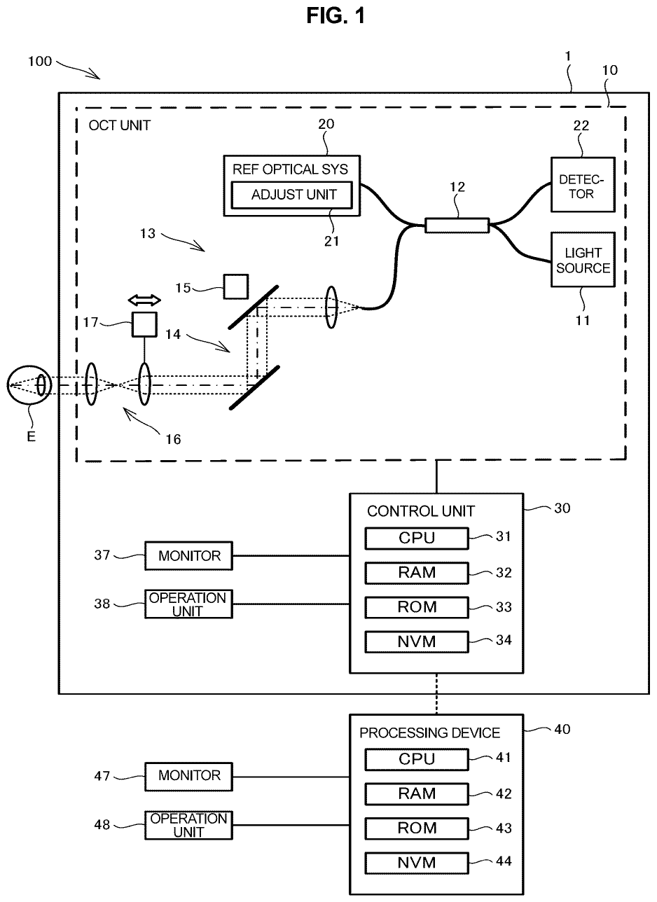

With reference to FIG. 1, a schematic configuration of a medical image processing system 100 in the present embodiment will be described. The medical image processing system 100 in the present embodiment includes an imaging device 1 and a medical image processing device 40. The imaging device (an OCT device in the present embodiment) 1 captures a three-dimensional image of the living tissue by receiving light from the living tissue. The medical image processing device 40 executes processing of image data captured by the imaging device 1. A PC is used in the medical image processing device 40 in the present embodiment. However, the device that may serve as the medical image processing device 40 is not necessarily limited to the PC. For example, the imaging device (the OCT device) 1 or the like may serve as the medical image processing device 40. When the imaging device 1 serves as the medical image processing device 40, the imaging device 1 can appropriately process captured three-dimensional images while capturing the three-dimensional images of a living tissue. Further, a mobile terminal device such as a tablet terminal device or a smartphone may serve as the medical image processing device 40. The control units of multiple devices (for example, the CPU of the PC and the CPU 31 of the imaging device 1 may cooperatively perform various processes.

The configuration of the imaging device 1 in the present embodiment will be described. The imaging device (the OCT device) 1 includes an OCT unit 10 and a control unit 30. The OCT unit 10 includes an OCT light source 11, a coupler (light divider) 12, a measurement optical system 13, a reference optical system 20, and a photodetector 22.

The OCT light source 11 emits light (OCT light) for acquiring image data. The coupler 12 divides the OCT light emitted from the OCT light source 11 into a measurement light and a reference light. Further, the coupler 12 of the present embodiment combines the measurement light reflected by the living tissue (the fundus of the subject eye E in the present embodiment) and the reference light generated by the reference optical system 20 to have the measurement light and the reference light interfere with each other. That is, the coupler 12 of the present embodiment also serves as both a branching optical element that branches the OCT light into the measurement light and the reference light and a combined wave optical element that combines the reflected light of the measurement light and the reference light. The configuration of at least one of the branching optical element and the combined wave optical element may be changed. For example, an element other than the coupler (for example, a circulator, a beam splitter, or the like) may be used.

The measurement optical system 13 guides the measurement light divided by the coupler 12 to the subject eye, and returns the measurement light reflected by the living tissue to the coupler 12. The measurement optical system 13 includes a scanning unit (scanner) 14, an irradiation optical system 16, and a focus adjustment unit 17. The scanning unit 14 is configured to scan by moving the measurement light in a two-dimensional direction intersecting the optical axis of the measurement light by a driving unit 15. In the present embodiment, two galvanometer mirrors capable of deflecting the measurement light in different directions from each other are used as the scanning unit 14. However, another device (for example, at least one of a polygon mirror, resonant scanner, acousto-optic element, or the like) that deflects light may be used as the scanning unit 14. The irradiation optical system 16 is located on a downstream side of the scanning unit 14 in an optical path (that is, the side close to the subject), and irradiates the living tissue with the measurement light. The focus adjustment unit 17 adjusts the focus of the measurement light by moving an optical element (for example, a lens) included in the irradiation optical system 16 in a direction along the optical axis of the measurement light.

The reference optical system 20 generates a reference light and returns the light to the coupler 12. The reference optical system 20 in the present embodiment generates the reference light by reflecting, by a reflective optical system (e.g., a reference mirror), the reference light divided by the coupler 12. However, the configuration of the reference optical system 20 may also be changed. For example, the reference optical system 20 may transmit light incident from the coupler 12 without reflecting the light and may return the light to the coupler 12. The reference optical system 20 includes an optical path length difference adjustment unit 21 that changes an optical path length difference between the measurement light and the reference light. In the present embodiment, the optical path length difference is changed by moving the reference mirror in the optical axis direction. The means for changing the optical path length difference may be provided in the optical path in the measurement optical system 13.

The photodetector 22 detects an interference signal by receiving the interference light of the measurement light and the reference light that are generated by the coupler 12. In this embodiment, the principle of Fourier domain OCT is used. In the Fourier domain OCT, the spectral intensity (the spectral interference signal) of the interference light is detected by the photodetector 22, and a complex OCT signal is acquired by the Fourier transform for the spectral intensity data. As an example of Fourier domain OCT, Spectral-domain-OCT (SD-OCT), Swept-source-OCT (SS-OCT), and the like can be used. Further, for example, it is possible to use Time-domain-OCT (TD-OCT) and the like.

Further, in the present embodiment, three-dimensional image data is acquired by scanning a spot of the measurement light in a two-dimensional region by the scanning unit 14. However, the principle of acquiring data for three-dimensional images may be changed. For example, three-dimensional image data may be acquired by the principle of line field OCT (hereinafter, referred to as “LF-OCT”). In LF-OCT, the measurement light is simultaneously emitted along an irradiation line extending in one-dimensional direction in a tissue, and the reflected light of the measurement light and the interference light of the reference light are received by a one-dimensional photodetector (for example, a line sensor) or a two-dimensional photodetector. In a two-dimensional measurement area, three-dimensional OCT data is acquired by scanning with the measurement light in a direction intersecting the irradiation line. Further, the three-dimensional image data may be acquired by the principle of full-field OCT. In the full-field OCT, a two-dimensional region on a tissue of the subject is simultaneously irradiated with measurement light, and the interference light is received by a two-dimensional photodetector.

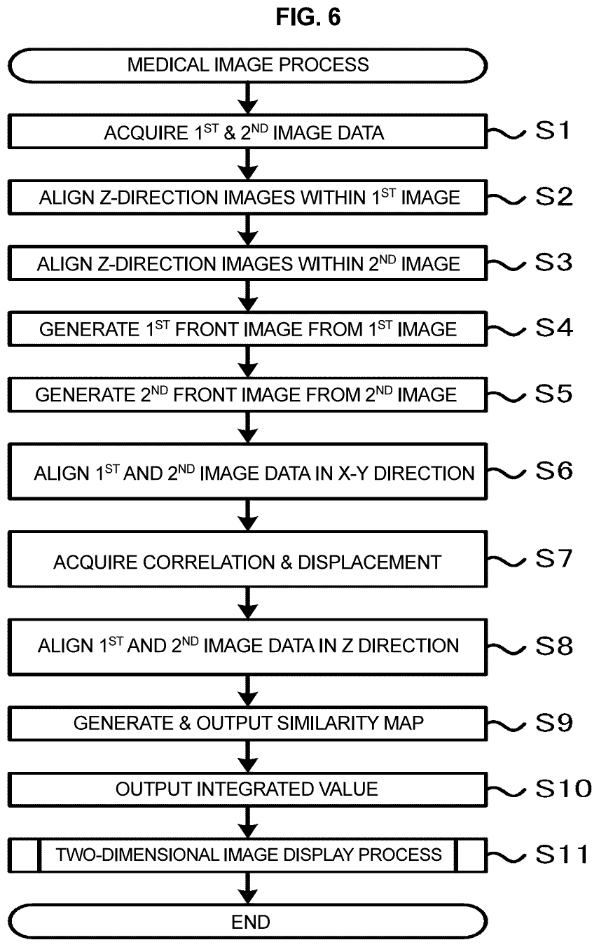

The control unit 30 performs various controls on the imaging device 1. The control unit 30 includes a CPU 31, a RAM 32, a ROM 33, and a non-volatile memory (NVM) 34. The CPU 31 is a controller that performs various controls. The RAM 32 temporarily stores various information. The ROM 33 stores programs to be executed by the CPU 31, various initial values, and the like. The NVM34 is a non-transient storage medium that is configured to keep storage contents even when a power supply is interrupted. When the imaging device 1 serves as a medical image processing device, a medical image processing program for executing a medical image process described later (see FIG. 6) may be stored in the NVM 34 or the like.

The monitor 37 and an operation unit 38 are connected to the control unit 30. The monitor 37 is one example of a display unit for displaying various images. The operation unit 38 is operated by a user in order for the user to input various operation instructions into the imaging device 1. For example, various devices such as a mouse, keyboard, touch panel, and foot switch can be used as the operation unit 38. Note that various operation instructions may be input into the imaging device 1 by inputting sound via a microphone.

A schematic configuration of the medical image processing device 40 will be described below. The medical image processing device 40 includes a CPU 41, a RAM 42, a ROM 43, and an NVM 44. A medical image processing program for performing the medical image process (see FIG. 6) described later may be stored in the NVM 44. Further, a monitor 47 and an operation unit 48 are connected to the medical image processing device 40. The monitor 47 is one example of a display unit for displaying various images. The operation unit 48 is operated by a user in order for the user to input various operation instructions into the medical image processing device 40. As the operation unit 48, various devices such as a mouse, a keyboard, and a touch panel can be used as with the operation unit 38 of the imaging device 1. Further, by inputting sound via a microphone 46, various operation instructions may be input into the medical image processing device 40.

The medical image processing device 40 acquires various data (for example, image data captured by the imaging device 1) from the imaging device 1. Various data may be acquired by, for example, at least one of wired communication, wireless communication, and a removable storage device (e.g., a USB memory), etc.

### Three-Dimensional Image

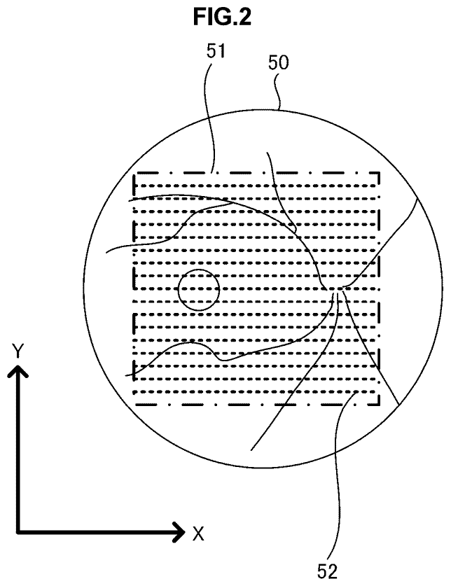

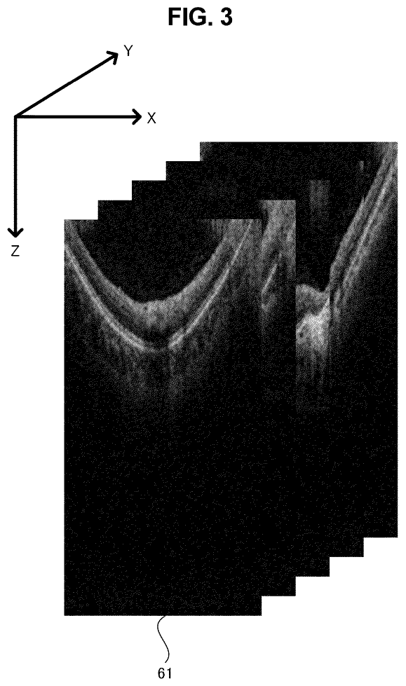

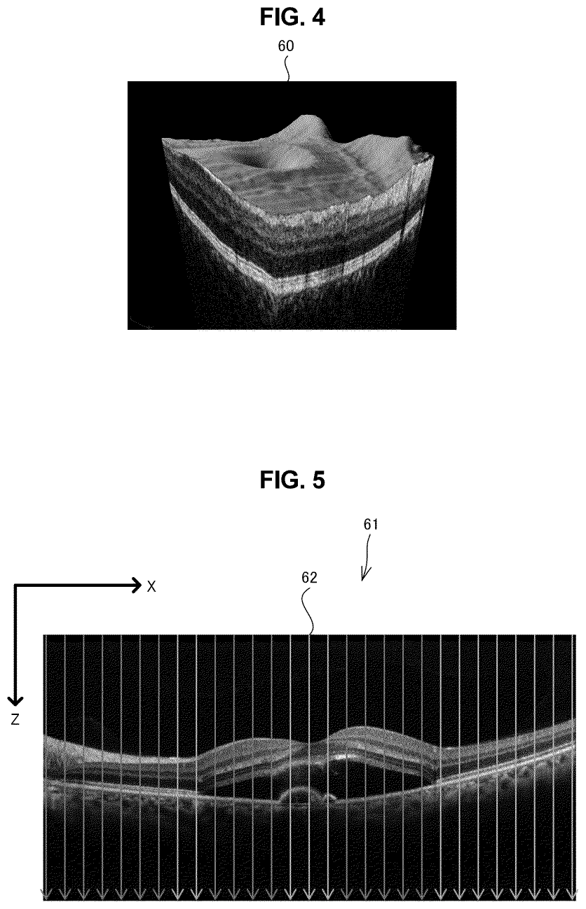

Referring to FIGS. 2 to 5, an example of a three-dimensional image as a processing target will be described. As shown in FIG. 2, the imaging device 1 in the present embodiment scans the two-dimensional imaging region 51 in the living tissue 50 (in the example shown in FIG. 2, a fundus tissue) with a light (the measurement light). Specifically, the imaging device 1 in the present embodiment emits light on scan lines 52 extending in a predetermined direction in the imaging region 51, thereby imaging (i.e., capturing) a two-dimensional image 61 (see FIG. 3) extending in Z direction along the optical axis of light and in X direction perpendicular to Z direction. In the example shown in FIG. 3, Z direction is a direction perpendicular to the two-dimensional imaging region 51 (in the depth direction of the living tissue 50), and X direction is a direction in which the scan lines 52 extend. Next, the imaging device 1 moves the position of the scan line 52 in Y direction on the imaging region 51 and repeats imaging (capturing) the two-dimensional image 61. Y direction is a direction that intersects both Z direction and X direction (perpendicularly intersecting in the present embodiment). As a result, a plurality of two-dimensional images 61 that each pass through each of the plurality of scan lines 52 and extend in the depth direction of the living tissue 50 are acquired. Then, as shown in FIG. 3, the plurality of two-dimensional images 61 are arranged in Y direction (in the direction of intersecting the imaging region of each two-dimensional image) to generate a three-dimensional image 60 (see FIG. 4) in the imaging region 51. That is, the target image data processed by the medical image processing device 40 in the present embodiment is image data of a three-dimensional image that spreads in Z direction that is the depth direction of the living tissue and the two-dimensional X-Y directions that intersects Z direction.

As shown in FIG. 5, focusing on one of the plurality of two-dimensional images 61 constituting the three-dimensional image, each two-dimensional image 61 is formed by arranging, in the X direction, a plurality of Z-direction images 62 each extending in the Z direction. As an example, the image processed in the present embodiment is an OCT image. In the present embodiment, each of the plurality of A-scan images forming the OCT image is a Z-direction image. As described above, the three-dimensional image is formed by arranging the plurality of two-dimensional images 61 in the Y direction. Therefore, the target image data processed by the medical image processing device 40 in the present embodiment is image data of a three-dimensional image formed by arranging, in the two-dimensional X-Y directions intersecting the Z direction, a plurality of Z-direction images 62 extending in the Z direction.

### Medical Image Processing

With reference to FIGS. 6 to 12, a medical image process executed by the medical image processing device 40 in the present embodiment will be described. In the present embodiment, the medical image processing device 40 which is a PCT acquires image data of a three-dimensional image of a living tissue from the imaging device 1 and processes the acquired image data. However, as described above, other devices may serve as the medical imaging device. For example, the imaging device (the OCT device in the present embodiment) 1 itself may execute the medical image process. Further, a plurality of control units (for example, the CPU 31 of the imaging device 1 and the CPU 41 of the medical image processing device 40) may collaboratively execute the medical image process. In the present embodiment, the CPU 41 of the medical image processing device 40 executes the medical image process shown in FIG. 6 according to the medical image processing program stored in the NVM 44.

First, the CPU 41 acquires first image data and second image data captured by the imaging device 1 (S1). The first image data and the second image data are generated by capturing a living tissue of the same subject at different timings. The imaging position (an imaging region) of the first image data and the imaging position of the second image data at least partially overlap with each other. If there is no change in the living tissue between the timings of capturing the first image data and the second image data, and both the first image data and the second image data are captured with high accuracy, the images in the overlapped imaging region between the first image data and the second image data are the same. On the other hand, if there is a change in the living tissue between the two timings of capturing the first image data and the second image data, an image of a part where the change occurred in the overlapped imaging region between the first image data and the second image data is different from an image of a part where the change did not occur.

For example, a user may operate the operation unit 48 and specify two pieces of image data as the first image data and the second image data to compare the two pieces of the image data for performing follow-up observation or the like. The two pieces of the image data are specified among from a plurality of image data captured at different timings for a particular subject. The CPU 41 may acquire the first image data and the second image data specified by the user.

At S1 in the present embodiment, two pieces of the image data captured on different dates are acquired as the first image data and the second image data. In this case, the process described below is performed so that the change in the living tissue between the two timings of capturing the first image data and the second image data is easily recognized. Therefore, follow-up observation on the living tissue and the like can be more appropriately performed. However, at S1, the first image data and the second image data captured at different timings on the same day may be acquired.

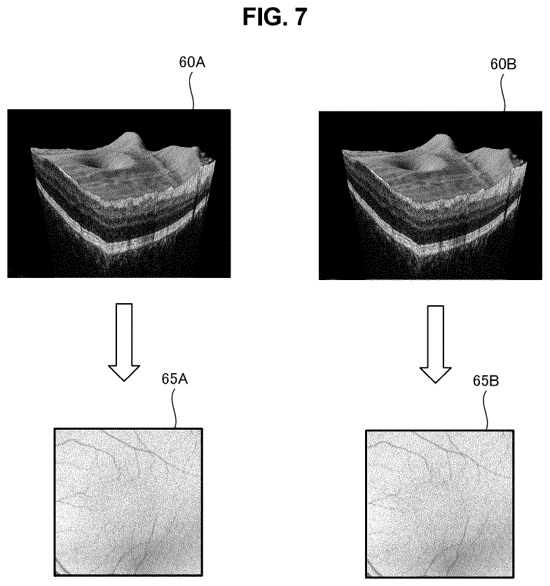

The CPU 41 aligns a plurality of Z-direction images 62 (see FIG. 5) within the three-dimensional first image 60A (see FIG. 7) of the first image data (S2). That is, the CPU 41 aligns, in the Z direction, the plurality of Z-direction images 62 included in the first image data. Similarly, the CPU 41 aligns a plurality of Z-direction images 62 within the three-dimensional second image 60B (see FIG. 7) of the second image data (S3). At least one of the plurality of Z-direction images 62 included in the three-dimensional image data (each of the first image data and the second image data) may include an image in which the position of the living tissue in the Z direction is deviated from the normal position due to the movement of the living tissue during imaging, vibration of the device, and the like. Since alignment between the plurality of Z-direction images 62 is performed in advance for each of the first image data and the second image data, the influence by the image whose position in the Z direction is deviated from the normal position is reduced. As a result, the accuracy of various processes performed thereafter, the accuracy of the output (displayed) image, and the like can be improved.

At S2 and S3, a specific method for aligning the plurality of Z-direction images 62 in each image data can be appropriately selected. For example, the CPU 41 may detect a particular layer or a layer boundary appearing in the three-dimensional image (each of the first and second images), and perform alignment of an image appearing in each of the plurality of Z-direction images 62 in the Z direction such that the detected layer or boundary linearly approaches along a particular direction (e.g., along an X-Y plane). Further, the CPU 41 may move each of the plurality of Z-direction images 62 in the Z direction such that the positions of portions each having a maximum brightness in the plurality of the Z-direction images 62 are aligned with each other in the Z direction. Alternatively, the CPU 23 may detect the amount of positional deviation (i.e., displacement) between the adjacent Z-direction images 62 using a phase-only correlation method or template matching, and then align the plurality of Z-direction images 62 in the Z direction so that the detected amount of positional deviation is eliminated.

Further, the amount of positional deviation in the Z direction between the plurality of Z-direction images 62 included in the three-dimensional image (each of the first image and the second image) may include a movement component due to the movement of the living tissue during capturing of the image and a shape component due to the shape of the living tissue. In this case, during the processing of S2 and S3, the CPU 41 performs a process for eliminating only the movement component (that is, a process of keeping only the shape component) among the amount of positional deviation between the plurality of Z-direction images 62. For the above processing, for example, the technology described in JP 2020-162886 A, which is incorporated herein by reference, may be used.

Then, as shown in FIG. 7, the CPU 41 generates, based on each of two three-dimensional images (the first image 60A and the second image 60B), two-dimensional images 65A, 65B generated by capturing the target living tissue in the Z direction (in the present embodiment, a direction along the optical axis of the measurement light) (S4, S5). That is, the CPU 41 generates a first two-dimensional front image 65A generated by capturing the target tissue in the Z direction based on the three-dimensional first image 60A (S4). Further, the CPU 41 generates a second two-dimensional front image 65B generated by capturing the target tissue in the Z direction based on the three-dimensional second image 60B (S5).

Details will be described later, but in the present embodiment, the first front image 65A and the second front image 65B are used to specify the same position in the X-Y directions between the first image 60A and the second image 60B. Further, in the present embodiment, when extracting a two-dimensional image from each of the first image 60A and the second image 60B, the first front image 65A and the second front image 65B are used, among the first image 60A and the second image 60B, to identify the same extraction position in which the positions in the living tissues are common to each other. That is, in the present embodiment, the front images 65A and 65B are generated from the first image data and the second image data from which the same position in the X-Y directions needs to be specified, and then the position is specified based on the generated front images 65A and 65B. Therefore, compared to another method (for example, a method for specifying a position based on front images captured by a principle different from the principle of capturing the first image data and the second image data), the position of the first image data and the position of the second image data in the X-Y directions can be identified more accurately.

A specific method for generating a front image (the first front image 65A and the second front image 65B) based on a three-dimensional image (the first image 60A and the second image 60B) can be appropriately selected. For example, the CPU 41 may generate the front images 65A and 65B by adding (or may be an additive average) the pixel values of a plurality of pixels arranged in the Z direction for each of the plurality of Z-direction images 62 in the image data. Further, the CPU 41 may generate the front images 65A and 65B (so-called, Enface images) based on pixel values at a specific position or range (for example, pixel values of a specific layer, pixel values of a specific boundary, among the layers and boundaries appearing in the image data, or pixel values within a specific layer/boundary range). In this case, the CPU 41 may execute a segmentation process to detect at least any of the layers/boundaries appearing in the image data, and generate the front images 65A and 65B based on the results of the segmentation process.

Next, the CPU 41 aligns the first front image 65A and the second front image 65B (S6). As a result, for the first image data and the second image data, a position in the X-Y direction in which the imaging positions of the living tissues are aligned with each other is specified. That is, in the present embodiment, by performing the alignment of the first front image 65A and the second front image 65B, the imaging positions of the living tissues of the first image data and the second image data are aligned with each other in all areas of the common imaging region in the X-Y direction. Thus, the positions of the first image data and the second image data in the X-Y directions can be more easily and accurately identified.

When aligning the first front image 65A and the second front image 65B, the CPU 41 may change the relative position and the relative angle between the two front images 65A, 65B while superimposing the first front image 65A with the second front image 65B. The CPU 41 may align the two front images 65A and 65B by searching for a position and angle at which the correlation between the two front images 65A and 65B has the highest value. Further, by using a method of an image local feature (for example, SIFT: Scale-Invariant Feature Transform) or of a rotational invariant phase limited correlation, the first front image 65A and the second front image 65B may be aligned.

Next, among the plurality of Z-direction images 62 included in each of the first image data and the second image data, the CPU 41 acquires, for positions in the X-Y direction, correlations and displacements (i.e., a deviation amount in the Z direction) between a plurality of pairs of the Z-direction image 62A in the first image data and the Z-direction image 62B in the second image data. Each pair of the Z-direction image 62A and the Z-direction image 62B has the same imaging position in the X-Y direction (S7). Among the positions in the X-Y direction, at a position where there is a small difference between the first image data and the second image data (for example, a position where the change between the living tissues is small, etc.), the correlation between the two Z-direction images 62 at the same position of the living tissues increases. On the other hand, in each position in the X-Y direction, at a position where there is a large difference between the first image data and the second image data (for example, a position where a large change occurs between the living tissues, etc.), the correlation between the two Z-direction images 62 at the same position of the living tissue decreases. Therefore, according to the correlation information acquired for each position in the X-Y direction, the difference between the first image data and the second image data (e.g., a change between the living tissues over time, etc.) can be appropriately recognized.

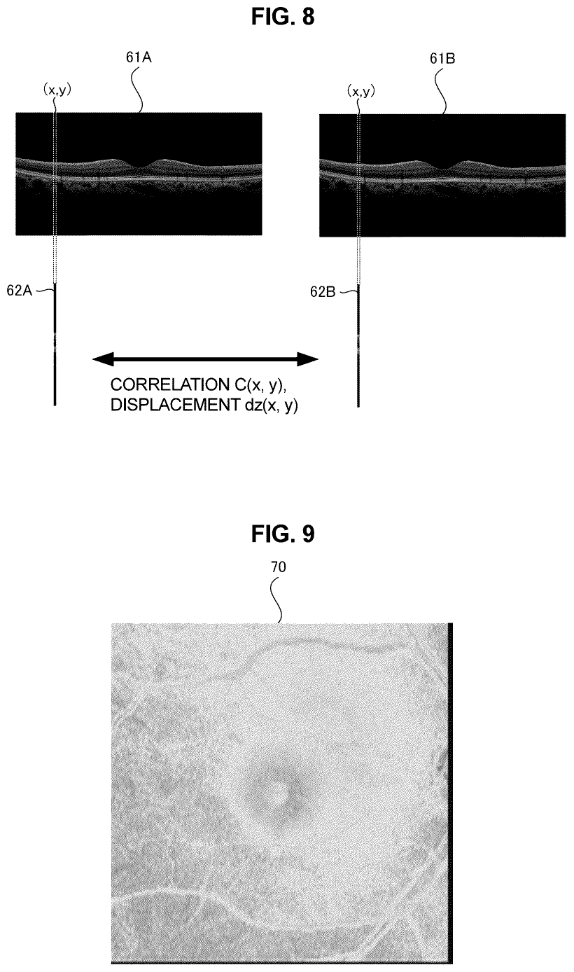

Referring to FIG. 8, one example of a method for acquiring correlation and displacement between the Z-direction images 62A and 62B at the same position in the X-Y directions will be described. As shown in FIG. 8, in the present embodiment, the CPU 41 identifies a Z-direction image 62A in the first image data and a Z-direction image 62B in the second image data in which the imaging positions of the living tissues in the X-Y direction are the same. As described above, in the present embodiment, alignment in the X-Y directions between the first image data and the second image data has been performed in advance. Thus, based on the result of the alignment, the same imaging positions of the living tissues in the X-Y direction between the Z-direction images 62A and 62B can be appropriately identified (that is, positions (x, y) in the example shown in FIG. 8). Next, the CPU 41 searches for a value of the highest correlation between the two Z-direction images 62A and 62B by changing the relative positions in the Z direction of the two Z-direction images 62A and 62B having the same imaging positions. The CPU 41 acquires the highest value of the searched correlation as a correlation C (x, y) between the two Z-direction images 62A and 62B. Further, the CPU 41 acquires the displacement dz (x, y) of the relative positions in the Z direction between the two Z-direction images 62A and 62B when the correlation between the two Z-direction images 62A and 62B has a highest value. The above-described process is performed for each of the plurality of positions in the X-Y directions. As a correlation acquisition method, at least one of normalized cross-correlation, correlation coefficient, phase correlation, or the like may be used. SSD (sum of squared errors), SAC (sum of absolute errors), or the like may be acquired as a correlation. In this case, the smaller the value, the higher the similarity between the two Z-direction images.

Next, the CPU 41 performs alignment between the first image data and the second image data in the Z direction for each Z-direction image 62 based on the displacement acquired at each position in the X-Y direction at S7 (that is, the displacement between the two Z-direction images 62A and 62B at the same position of the living tissue) (S8). As a result, the first image data and the second image data can be aligned more accurately.

Next, the CPU 41 executes a generation process to generate a similarity map 70 (see FIG. 9) that shows a two-dimensional distribution in the X-Y directions of the strength of the correlations acquired for the positions in the X-Y directions. Then, the CPU 41 outputs the generated similarity map 70 (S9). By checking the similarity map 70, a user can intuitively recognize the two-dimensional distribution in the X-Y direction of the strength of the correlations between the first image data and the second image data. For example, by checking the similarity map 70, a user can appropriately recognize the position in the X-Y direction at which the living tissue is likely to have a large change over time.

At S9 in the present embodiment, the CPU 41 acquires the correlations at the positions in the X-Y direction between the entire captured first image data and the second image data (that is, at least the entire imaging region overlapping between the first and second image data). Therefore, the difference between the first image data and the second image data is appropriately recognized throughout the imaging region of the overlapping images. Therefore, the user can be appropriately assisted in medical treatment.

The method for outputting the similarity map 70 can be selected as appropriate. For example, the CPU 41 may output the similarity map 70 by displaying the similarity map 70 on the monitor (i.e., the display unit) 47. In this case, the CPU 41 may show a two-dimensional distribution of the strength of the correlation by changing at least one of colors, brightness, etc. at each position of the similarity map 70 according to the strength of the correlation. Further, the CPU 41 may output the data of the generated similarity map 70 to another device. Note that the CPU 41 may display the similarity map 70 after performing a smoothing process on, or a process of applying a minimum value filter to, the correlation values acquired for each position in the X-Y direction. In this case, a position with a large difference between the first image data and the second image data can appropriately appear on the similarity map 70.

Further, the CPU 41 can collectively or switchably display a deviation map and the correlation map 70 on the monitor 47. The deviation map indicates a two-dimensional distribution of deviation of the actually acquired probability distribution relative to a probability distribution that would be generated when layers or a boundary of a target can be identified accurately when a mathematical model trained by a machine learning algorithm identifies at least one of the layers and boundaries appearing in the three-dimensional image. In this case, the CPU 41 acquires a probability distribution for identifying at least one of the layers or the boundary of a fundus tissue appearing in the three-dimensional image by inputting at least one of the first image data and the second image data (e.g., data captured immediately before) into a mathematical model trained by a machine learning algorithm. The CPU 41 generates the deviation map based on the acquired probability distribution. As described above, the degree of abnormality in the structure of layers and boundaries is likely to be shown in the deviation map. Further, according to the correlation map 70, the position in the X-Y directions, which is likely to have a large change over time in the living tissue, is easily recognized. Therefore, by collectively or switchably displaying the deviation map and the correlation map 70 on the monitor 47, it is easier for the user to recognize the status of the living tissue of the subject. In addition, for the method for acquiring the degree of deviation, the method for generating the degree of deviation, and the like, the technology described in JP 2020-018794 A, which is incorporated herein by reference, can be used.

Further, the CPU 41 can collectively or switchably display a thickness map and the correlation map 70 on the monitor 47. The thickness map shows a two-dimensional distribution of analysis results on the thickness of at least one of the layers and boundaries appearing in the three-dimensional image. In this case, the CPU 41 generates the thickness map based on at least one of the first image data and the second image data. According to the thickness map, the distribution of the thickness of the layers of the living tissue is properly recognized. Further, as described above, according to the correlation map 70, the position in the X-Y directions, which is likely to have a large change over time in the living tissue, is easily recognized. Therefore, by collectively or switchably displaying the thickness map and the correlation map 70 on the monitor 47, it is easier for the user to recognize the status of the living tissue of the subject.

The CPU 41 calculates an integrated value of the plurality of correlations acquired for positions in the X-Y direction, and outputs the calculated integrated value of the correlations (S10). The user can appropriately recognize the difference between the first image data and the second image data by the integrated value of the correlations. For example, the user can recognize, by the integrated value of the correlations, the degree of overall change in the living tissue over time between the time at which the first image data was captured and the time at which the second image data was captured. Further, it is also possible to evaluate, by the integrated value of the correlations, the accuracy of the alignment of the first image data and the second image data at the time of acquiring the correlation between the Z-direction images 62. Further, if the accuracy of imaging of at least one of the first image data and the second image data is low, the integrated value of the correlations is small. Therefore, it is also possible to evaluate the accuracy of imaging based on the integrated value of the correlations. For example, when the second image data is newly captured, if the integration value of the correlations between the first image data and the second image data is less than a threshold value, the CPU 41 determines that the accuracy of imaging the second image data is low. Then, the CPU 41 may output an instruction for re-capturing the second image data.

In addition, the method for outputting the calculated integrated value of the correlations can also be appropriately selected. In the present embodiment, the CPU 41 calculates an average value of the plurality of correlations as an integrated value. However, the values of a plurality of correlations may be integrated by calculating the median, maximum, minimum, and the like of the plurality of correlations. The CPU 41 may output the calculated integrated value of the correlations or an indicator indicating the magnitude of the integrated value of the correlations (for example, an indicator indicating which stage among a plurality of stages the magnitude of the calculated integrated value of the correlations corresponds to) by at least one of displaying on the display unit and outputting audio sound. Further, the CPU 41 may calculate, as an integrated value, the number or percentage of correlations each having a value greater than the threshold value, or the number or percentage of correlations each having a value less than the threshold value.

Next, the CPU 41 executes a two-dimensional image display process (S11) and terminates the process. At the two-dimensional image display process, the CPU 41 processes the three-dimensional first image data and the three-dimensional second image data so that a two-dimensional image useful for medical treatment by the user can be displayed on the monitor 47. In particular, in the present embodiment, the CPU 41 can automatically extract two-dimensional images (that is, the first two-dimensional image 61A and the second two-dimensional image 61B shown in FIG. 12) at the same position in the living tissue commonly captured in the images among the first image data and the second image data. By combining the first two-dimensional image 61A and the second two-dimensional image 61B extracted at the same position after the images 61A and 61B are aligned (that is, in a superimposed state), the CPU 41 generates a composite two-dimensional image 69 (see FIG. 12) and displays the generated image 69 on the monitor 47.

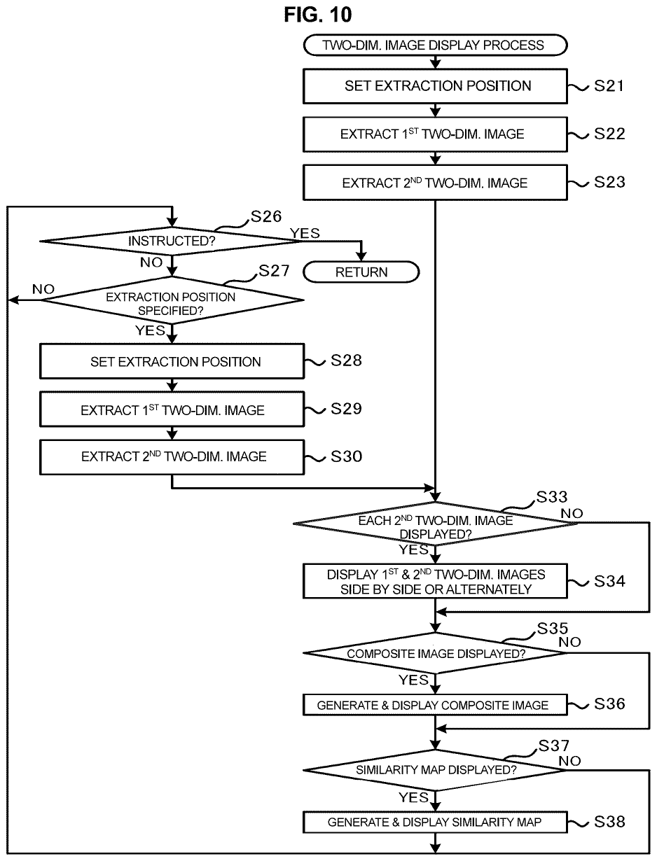

With reference to FIG. 10, the two-dimensional image display process will be described in detail. First, a process (S21-S23 and S33-S38) for automatically extracting and displaying a two-dimensional image at a position in which the correlation between the first image data and the second image data is low will be described.

The CPU 41 automatically sets the extraction position of the two-dimensional image 61 to a position at which the correlation acquired at S7 (see FIG. 6) has a lowest value (that is, the lowest correlation position 55 on the similarity map 70) among the imaging region of the first image data and the second image data spreading in the X-Y direction (S21). In this case, a user can recognize, by the two-dimensional image 61, the status of the living tissue (e.g., changes in the living tissue over time) at a position where the correlation between the first and second image data has a lowest value by checking the two-dimensional image 61 at the automatically set extraction position.

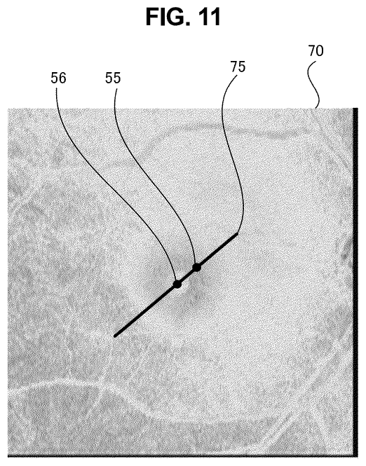

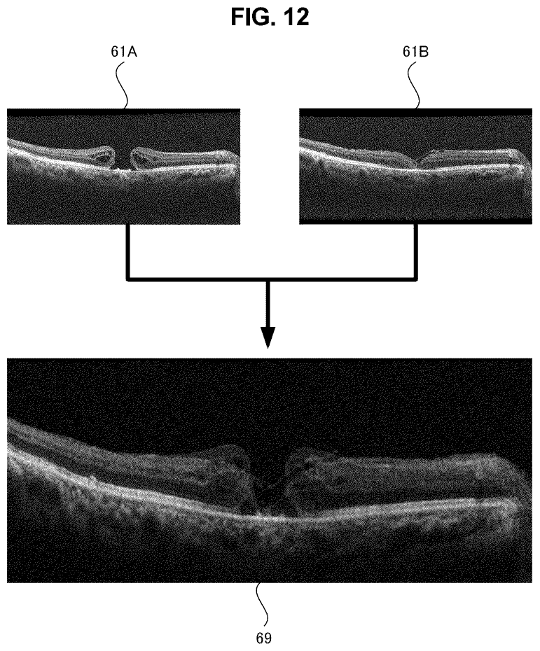

As shown in FIG. 11, in the present embodiment, the CPU 41 sets a linear extraction position 75 on the two-dimensional similarity map 70 extending in the X-Y directions. In the example shown in FIG. 11, a linear-shaped extraction position 75 is automatically set. However, a shape other than a straight line (for example, a curved line, an annular line, etc.) may be used as the shape of the extraction position 75. In the example shown in FIG. 11, the CPU 41 automatically sets the extraction position 75 to a linear-shaped position that passes through both the position 55 with the lowest correlation and a reference position 56 on the image (e.g., a macular center as shown in FIG. 11). In this case, the CPU 41 may detect the reference position 56 on the image by performing publicly-known image processing on at least one of the three-dimensional image and the front image. Further, the CPU 41 may search for a position where the cumulative value of correlation at the extraction position in a predetermined shape (for example, a line, etc.) is smallest, and automatically set the searched position as the extraction position. Further, the CPU 41 may automatically set the extraction position at a linear-shaped position having a center at the position where the correlation is lowest and extending in a predetermined direction (for example, in the X direction or the Y direction, etc.).

The CPU 41 extracts, from the first image data, a first two-dimensional image 61A (see FIG. 12) that passes through the extraction position 75 automatically set at S21 and spreads in the Z direction (S22). Further, the CPU 41 extracts, from the second image data, a second two-dimensional image 61B (see FIG. 12) that passes through the extraction position 75 automatically set at S21 and spreads in the Z direction (S23). That is, at S22 and S23, the CPU 41 extracts, among from the first image data and the second image data, the two-dimensional images 61A and 61B at the same extraction position 75 where the positions in the living tissues appearing in the images are the same as each other.

As described above, in the present embodiment, the front images 65A and 65B are generated from the first image data and the second image data from which the same extraction position 75 in the X-Y directions needs to be specified, and then the extraction position 75 of each of the two-dimensional images 61A, 61B is specified based on the generated front images 65A and 65B. Therefore, as compared to another method (for example, a method for specifying an extraction position based on front images captured by a principle different from the principle of capturing the first image data and the second image data), the extraction position of the two-dimensional image 61A, 61B of the first image data and the second image data in the X-Y direction can be identified more accurately.

As described above, in the present embodiment, by aligning the first front image 65A and the second front image 65B, the same extraction position 75 in the first image data and the second image data in the X-Y direction is specified. As a result, the positions of the first image data and the second image data in the X-Y direction on the living tissue are aligned with each other in the entire range within the common imaging region in the X-Y direction. Thus, the extraction positions 75 of the first image data and the second image data can be more easily and accurately identified.

The CPU 41 combines the first two-dimensional image 61A and the second two-dimensional image 61B and displays them on the monitor 47 after the images appearing in each of the first two-dimensional image 61A and the second two-dimensional image 61B were aligned with each other in the Z direction. Therefore, the first two-dimensional image 61A and the second two-dimensional image 61B extracted from the same extraction position 75 in the X-Y direction are combined and displayed in a state where alignment in the Z direction is also performed. Thus, the user can appropriately compare each subject in the first two-dimensional image 61A and the second two-dimensional image 61B by confirming the composite two-dimensional image 69. In the present embodiment, the CPU 41 extracts the first two-dimensional image 61A and the second two-dimensional image 61B after aligning in the Z direction in advance between the first image data and the second image data, each of which is a three-dimensional image. Therefore, regardless of the extraction position, the relative positions of the extracted first two-dimensional image 61A and the second two-dimensional image 61B in the Z direction are appropriately fixed.

When displaying the extracted two-dimensional images 61A and 61B (S33: YES), the CPU 41 displays the first two-dimensional image 61A and the second two-dimensional image 61B extracted at S22 and S23 side by side or by switching on the monitor 47 (S34). In this case, the user can appropriately compare the two-dimensional images 61A and 61B at the same extraction position in which the positions in the living tissue appearing in the image are common to each other among the first image data and the second image data. Therefore, changes in the living tissue over time can be easily recognized appropriately by comparing the two two-dimensional images 61A and 61B.

When displaying the composite two-dimensional image 69 (see FIG. 12) (S35: YES), the CPU 41 combines the first two-dimensional image 61A and the second two-dimensional image 61B extracted at the same position after the two two-dimensional images 61A and 61B were aligned with each other to generate the composite two-dimensional image 69. Then, the CPU 41 displays the generated composite image 69 on the monitor 47 (S36). By confirming the composite two-dimensional image 69 displayed on the monitor 47, the user can more appropriately compare the subjects of the first two-dimensional image 61A and the second two-dimensional image 61B, which have a common imaging position in the living tissue.

A method for displaying the composite two-dimensional image 69 on the monitor 47 will be described in detail. In the present embodiment, the CPU 41 combines and displays the first two-dimensional image 61A and the second two-dimensional image 61B in different colors from each other. As a result, in one composite two-dimensional image 69, the subject appearing in the first two-dimensional image 61A and the subject appearing in the second two-dimensional image 61B are displayed in different colors. Therefore, the user can further appropriately recognize the difference in the living tissues appearing in the first two-dimensional image 61A and the second two-dimensional image 61B (for example, changes in the living tissues over time between the time of acquisition of the first image data and the time of acquisition of the second image data, etc.) based on the composite two-dimensional image 69.

Further, the CPU 41 sets the color of pixels in the background (i.e., background color) in which the subject does not appear in the composite two-dimensional image 69 to a black color or a white color. In the first two-dimensional image 61A, the color of the pixel of the part where the subject appears is referred to as a first color. In the second two-dimensional image 61B, the color of the pixel of the part where the subject appears is referred to as a second color. When displaying the composite two-dimensional image 69, the CPU 41 sets the combination of the first color and the second color to a combination of colors that will turn to be opposite colors to the background color (i.e., black or white) when the first and second colors are combined. As a result, at a position where the subject of the first two-dimensional image 61A and the subject of the second two-dimensional image 61B overlap with each other, the subject is displayed in a color (white or black) opposite to the background color. That is, at a position where the subject of the first two-dimensional image 61A and the subject of the second two-dimensional image 61B overlap with each other, the subject is displayed in grayscale. Further, at a position where the subject of the first two-dimensional image 61A and the subject of the second two-dimensional image 61B do not overlap with each other, the subject of the first two-dimensional image 61A is displayed in the first color, and the subject of the second two-dimensional image 61B is displayed in the second color (a color different from the first color). As a result, at a position where the subject of the first two-dimensional image 61A and the subject of the second two-dimensional image 61B do not overlap with each other, the subject of each two-dimensional image 61A and 61B is appropriately recognized with the first and second colors other than grayscale. Therefore, the user can intuitively recognize the portion where only the subject of the first two-dimensional image 61A appears, the portion where only the subject of the second two-dimensional image 61B appears, the part where the subject of the first two-dimensional image 61A and the subject of the second two-dimensional image 61B overlap with each other, and the background portion.

In other words, if the composite color of the first color and the second color does not turn to be an opposite color to the background, the first color, the second color, and the composite color would be included in the composite two-dimensional image. In this case, the visibility of the subject tends to decrease. For example, if the first color is red and the second color is green, yellow, which is a composite color of red and green, would be also included in the composite two-dimensional image 69. On the contrary, by using the composite color of the first color and the second color which is an opposite color to the background color, the position where the two subjects overlap with each other is displayed in grayscale, and the visibility of the subject can be improved.

As one example, when the background color is black, the CPU 41 uses a composite color of two of the three primary colors (Red, Green, Blue) of light that will turn to be white when all colors are combined as one of the first and second colors and uses a remaining color as the other of the first and second colors. Further, when the background color is white, the CPU 41 uses a combined color of two of the three primary colors (Cyan, Magenta, Yellow) of light that will turn to be black when all colors are combined as the first color and uses a remaining color as the second color. However, it is also possible to change the combination of the first and second colors.

Further, the CPU 41 may display a two-dimensional image similarity map showing a two-dimensional distribution of similarity between the first two-dimensional image 61A and the second two-dimensional image 61B on the monitor 47. When generating the two-dimensional image similarity map, the control unit may calculate, for each partial image at each position, a similarity between partial images where the first two-dimensional image and the second two-dimensional image are at the same position by alignment among a plurality of partial images constituting each of the first and second two-dimensional images. The CPU 41 generates the two-dimensional image similarity map based on the similarity calculated for the partial image at each position. When displaying the two-dimensional image similarity map (S37: YES), the CPU 41 generates the two-dimensional image similarity map and displays it on the monitor 47 (S38). Thereafter, the process proceeds to S26. By checking the output two-dimensional image similarity map, the user can intuitively recognize the distribution of similarity when alignment of the first two-dimensional image 61A and the second two-dimensional image 61B is performed. Therefore, the user can better recognize the change in the living tissue over time between the time of capturing the first image data and the time of capturing the second image data.

A specific method for generating the two-dimensional image similarity map can be appropriately selected. For example, the two-dimensional image similarity map may be generated by using a known SSIM (Structural Similarity). In this case, the SSIM is calculated in a small area including a plurality of pixels. A plurality of SSIMs are calculated while shifting the relative positions of the two sub-regions, and the average value of the calculated plurality of SSIMs is considered to be the degree of similarity. As a result, in addition to the information of a pair of partial images at the same position (for example, luminance and contrast, etc.), information on the surrounding partial images is also used, so that the difference in the structure of the image can be evaluated. Further, the two-dimensional image similarity map may be generated by using a known DP matching method. In this case, changes in the subject appearing in the two-dimensional images 61A and 61B (for example, changes in layer thickness, etc.) are further easily recognized.

Next, the extraction positions 75 of the first two-dimensional image 61A and the second two-dimensional image 61B are set according to an instruction input by a user, and the process of displaying a two-dimensional image at the set extraction position (S26˜S30, S33˜S38) will be described.

When the CPU 41 does not receive an instruction to terminate the process (S26: NO), the CPU 41 determines whether the extraction positions of the two-dimensional images 61A and 61B are specified on the similarity map 70 (see FIGS. 9 and 11) displayed on the monitor 47 (S27). If not specified (S27: NO), the steps of S26 and S27 are repeated to be in a waiting state. When an instruction to terminate the process is input (S26: YES), the medical image process is terminated.

In the present embodiment, the CPU 41 accepts an instruction for specifying the extraction positions of the two-dimensional images 61A and 61B from a user on the similarity map 70 displayed on the monitor 47. As an example, in the present embodiment, the instruction for specifying the extraction position 75 is accepted by allowing the user to specify the linear-shaped extraction position 75 on the two-dimensional similarity map 70. The user specifies the linear-shaped extraction position 75 by operating the operation unit 48. As described above, various shapes such as a straight line, a curved shape, and an annular shape can be used as the shape of the line indicating the extraction position 75. Needless to say, a plurality of extraction positions 75 may be specified.

When the extraction positions of the two-dimensional images 61A and 61B are specified on the similarity map 70 (S27: YES), the CPU 41 sets the extraction positions 75 at the positions specified on the similarity map 70 (S28). As with the step of S22, the CPU 41 extracts, from the first image data, a first two-dimensional image 61A (see FIG. 12) that passes through the extraction position 75 automatically set at S28 and spreads in the Z direction (S29). Further, as with the step of S23, the CPU 41 extracts, from the second image data, a second two-dimensional image 61B (see FIG. 12) that passes through the extraction position 75 automatically set at S28 and spreads in the Z direction (S30). That is, at S29 and S30, the CPU 41 extracts, among from the first image data and the second image data, the two-dimensional images 61A and 61B at the same extraction position 75 where the positions in the living tissues appearing in the images are the same as each other. Thereafter, the steps of S33-S38 are executed. In this case, the user recognizes the two-dimensional distribution of the strength of the correlation between the first image data and the second image data indicated by the similarity map 70, and then has the two-dimensional image (at least one of the first two-dimensional image 61A and the second two-dimensional image 61B) at an appropriate position displayed. For example, at the position where the correlation indicated by the similarity map 70 is low, there is a high possibility that the greater change in the living tissue over time occurs between the first image data and the second image data. Therefore, by specifying the extraction position 75 at a position where the correlation is low in the similarity map 70, the user can appropriately recognize, based on two-dimensional image, the change in the living tissue over time.

The technology disclosed in the above embodiments is only one example. Thus, it is also possible to change the technique illustrated in the above embodiment. First, only a part of the processes exemplified in the above-described embodiment may be executed. For example, the CPU 41 may perform a process of displaying a composite two-dimensional image 69 without performing an output process of correlation (for example, a similarity map 70) between the first image data and the first image data. In this case, the extraction positions 75 of the first two-dimensional image 75 and the second two-dimensional image 75 may not be specified on the similarity map 70. For example, the CPU 41 may accept an instruction for specifying the extraction position 75 by the user on the front images 65A and 65B. Further, the CPU 41 may perform an output process of correlation between the first image data and the first image data without executing the process of displaying the composite two-dimensional image 69. In this case, the CPU 41 may extract and display only one of the first two-dimensional image 61A and the second two-dimensional image 61B from the image data.

Hereinafter, each step of the first aspect of the present disclosure corresponds to the processing of the above-described embodiment. The process of acquiring image data at S1 of FIG. 6 is one example of an “image data acquisition step”. The process of acquiring correlation at S7 in FIG. 6 is one example of a “correlation acquisition step”. The process of processing and outputting correlation at S9 and S10 of FIG. 6 is one example of a “correlation output step”. The process of receiving an instruction for specifying the extraction position of the two-dimensional image at S27 of FIG. 10 is one example of an “extraction position reception step”. The process of extracting and displaying a two-dimensional image at S22, S23, S29, S30, S33-S36 of FIG. 10 is one example of an “extraction display step”. The process of automatically setting the extraction position of the two-dimensional image at S21 of FIG. 10 is one example of an “automatic extraction position setting step”. The process of generating a first front image at S4 of FIG. 6 is one example of a “first front image generation step”. The process of generating a second front image at S5 of FIG. 6 is one example of a “second front image generation step”. The process of aligning in the Z direction in the first image data at S2 of FIG. 6 is one example of a “first image alignment step”. The process of aligning the second image data in the Z direction at S3 of FIG. 6 is one example of a “second image alignment step”.

Hereinafter, each step of the second aspect of the present disclosure corresponds to the processing of the above-described embodiment. The process of acquiring a two-dimensional image at S22, S23, S29, and S30 of FIG. 10 is one example of a “two-dimensional image acquisition step”. The process of displaying a composite two-dimensional image at S36 of FIG. 10 is one example of a “composite display step”. The process of acquiring image data at S1 of FIG. 6 is one example of an “image data acquisition step”. The process of setting the extraction position of the two-dimensional image at S21 and S28 of FIG. 10 is one example of an “extraction position setting step”. The process of outputting a two-dimensional image similarity map at S38 of FIG. 10 is one example of a “two-dimensional image similarity map output step”. The process of generating a first front image at S4 of FIG. 6 is one example of a “first front image generation step”. The process of generating a second front image at S5 of FIG. 6 is one example of a “second front image generation step”.

1. A medical image processing device that processes image data of a living tissue, the image data being image data of a three-dimensional image formed by arranging, in X-Y directions which are two-dimensional directions intersecting a Z direction, a plurality of Z-direction images each extending in the Z direction which is a depth direction of the living tissue, the medical image processing device comprising:
a control unit that is configured to execute:
an image data acquisition step of acquiring first image data and second image data that were captured on a living tissue of a same subject at different times, at least some of imaging positions for the first and second image data overlapping with each other;
a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and
a correlation output step of processing and outputting the plurality of correlations for the positions in the X-Y directions.

2. The medical image processing device according to claim 1, wherein
at the correlation output step, the control unit is further configured to:
generate a similarity map indicating a two-dimensional distribution in the X-Y directions of strength of the correlations acquired for the positions in the X-Y directions; and
output the generated similarity map.

3. The medical image processing device according to claim 2, wherein
the control unit is further configured to execute:
an extraction position reception step of receiving, from a user, an instruction for specifying an extraction position of a two-dimensional image on the similarity map displayed on a display unit; and
in response to receiving the instruction for specifying the extraction position at the extraction position reception step, an extraction display step of
extracting, from at least one of the first image data and the second image data, a two-dimensional image that passes through the specified extraction position and spreads in the Z direction, and
displaying the extracted two-dimensional image on the display unit.

4. The medical image processing device according to claim 3, wherein
at the extraction display step, the control unit is further configured to:
extract, from the first image data and the second image data, two-dimensional images each of which passes through the extraction position specified at the extraction position reception step and spreads in the Z direction; and
display the extracted two-dimensional images on the display unit.

5. The medical image processing device according to claim 2, wherein
the control unit is further configured to execute:
an extraction position automatic setting step of automatically setting, on the similarity map, an extraction position of a two-dimensional image to a position that includes a point having a lowest correlation; and
an extraction display step of
extracting, from at least one of the first and second image data, the two-dimensional image that passes through the extraction position automatically set at the extraction position automatic setting step and spreads in the Z direction, and
displaying the extracted two-dimensional image on a display unit.

6. The medical image processing device according to claim 5, wherein
at the extraction display step, the control unit is further configured to:
extract, from the first image data and the second image data, two-dimensional images each of which passes through the extraction position automatically set at the extraction position automatic setting step and spreads in the Z direction; and
display the two-dimensional images on the display unit.

7. The medical image processing device according to claim 1, wherein
at the correlation output step, the control unit is further configured to:
integrate a plurality of values of the correlations acquired for the positions in the X-Y directions; and
output an integrated value of the correlations.

8. The medical image processing device according to claim 1, wherein
the control unit is further configured to execute:
a first front image generation step of generating, based on the first image data, a two-dimensional first front image that is an image of a target subject when viewed in the Z direction; and
a second front image generation step of generating, based on the second image data, a two-dimensional second front image that is an image of the target subject when viewed in the Z direction, and
at the correlation acquisition step, the control unit is further configured to acquire each of the correlations by specifying, based on the first front image and the second front image, the pair of the one of the plurality of Z-direction images of the first image data and the corresponding one of the plurality of Z-direction images of the second image data.

9. The medical image processing device according to claim 1, wherein
the control unit is further configured to execute:
a first image alignment step of aligning, in the Z direction, between the plurality of Z-direction images included in the first image data; and
a second image alignment step of aligning, in the Z direction, between the plurality of Z-direction images included in the second image data.

10. The medical image processing device according to claim 1, wherein
at the correlation acquisition step, the control unit is further configured to acquire the correlations for the positions in the X-Y directions between the entire first image data and the entire second image data.

11. The medical image processing device according to claim 1, wherein
a date on which the first image data was captured is different from a date on which the second image data was captured.

12. An OCT device that captures image data of a living tissue by receiving interference light between reference light and reflected light of measurement light emitted onto the living tissue, the image data being image data of a three-dimensional image formed by arranging, in X-Y directions which are two-dimensional directions intersecting a Z direction, a plurality of Z-direction images each extending in the Z direction which is a depth direction of the living tissue, the OCT device comprising:
a control unit that is configured to execute:
an image data acquisition step of acquiring first image data and second image data that were captured on a living tissue of a same subject at different times, at least some of imaging positions for the first and second image data overlapping with each other;
a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and
a correlation output step of processing and outputting the plurality of correlations for the positions in the X-Y directions.

13. A non-transitory, computer readable, storage medium storing a medical image processing program for a medical image processing device that processes image data of a living tissue, the image data being image data of a three-dimensional image formed by arranging, in X-Y directions which are two-dimensional directions intersecting a Z direction, a plurality of Z-direction images each extending in the Z direction which is a depth direction of the living tissue, the program, when executed by a control unit of the medical image processing device, causing the control unit to execute:
an image data acquisition step of acquiring first image data and second image data that were captured on a living tissue of a same subject at different times, at least some of imaging positions for the first and second image data overlapping with each other;
a correlation acquisition step of acquiring, for positions in the X-Y directions, a plurality of correlations between the plurality of Z-directions images in the first image data and the plurality of Z-direction images in the second image data, each of the correlations being acquired for a pair of one of the plurality of Z-direction images in the first image data and a corresponding one of the plurality of Z-direction images in the second image data both of which have a same imaging position; and
a correlation output step of processing and outputting the plurality of correlations for the positions in the X-Y directions.
